(19) United States
(12) Patent Application Publication
(10) Pub. No.: US 2023/0340376 A1
(43) Pub. Date: Oct. 26, 2023

(54) HYGIENIC ODOR REDUCING AGENT IN SANITARY AREAS

(71) Applicant: Margaret Eboso Busisa, KITALE, RIFT VALLEY (KE)

(72) Inventor: Margaret Eboso Busisa, KITALE, RIFT VALLEY (KE)

(21) Appl. No.: 17/779,329

(22) PCT Filed: Sep. 23, 2020
(86) PCT No.: PCT/KE2020/050002

§ 371 Date: May 24, 2022

(30) Foreign Application Priority Data
Sep. 24, 2019 (KE) KE/P/2019/003381

(51) Int. Cl. C11D 7/30; C11D 17/00; C11D 7/06; C11D 7/12; A61L 9/012; C11D 11/00

(52) U.S. Cl. CPC C11D 7/06; A61L 9/012; C11D 7/12; C11D 7/30; C11D 11/0094; C11D 17/0008; A61L 2101/06

(57) ABSTRACT

A hygienic odor reducing agent in sanitary areas is provided which comprises chlorine, caustic soda, soda ash, chloroform and tide scent. The instant application also discloses an agent comprises 0.5 kilograms of chlorine in powder form and constituting 65% of the solution. In one embodiment, the invention discloses a hygienic odor reducing agent, characterized in that it comprises 0.25 kilograms of caustic soda in powder form, 0.25 kilograms of soda ash, 1 ml of chloroform and 5 ml of tide scent. Further disclosed is a process of preparing the hygienic odor reducing agent in sanitary areas which comprises the steps of pouring 10 liters of water in a container, adding 0.5 kilograms of chlorine, adding 0.25 kilograms of caustic soda, adding 0.25 kilograms of soda ash, adding 1 ml of chloroform, adding 5 ml of tide scent and stirring the whole mixture daily for 3 days.

### FIELD OF INVENTION

The present invention relates to hygienic odor reduction in sanitary areas comprising toilets, composts, sewerage and kitchen sinks, among others. These present invention relates to a solution product that cartelizes odor to the extent of having no smell in sanitary areas, and is useful as a disinfectant as well as an agent.

### BACKGROUND ART

From the observation in many institutions, odor is a very nagging situation and for that reason, a solution is needed and therefore, this influenced me to embark on this invention.

### SUMMARY OF THE INVENTION

This invention disclose a hygienic odor reducing agent in sanitary areas comprising chlorine, caustic soda, soda ash, chloroform and tide scent.

I also discloses an agent comprises 0.5 kilograms of chlorine in powder form and constituting 65% of the solution. In one embodiment, the invention discloses a hygienic odor reducing agent, characterized in that it comprises 0.25 kilograms of caustic soda in powder form, 0.25 kilograms of soda ash, 1 ml of chloroform and 5 ml of tide scent.

The invention also disclose a process of preparing the hygienic odor reducing agent in sanitary areas comprising the steps of, pouring 10 liters of water in a container, adding 0.5 kilograms of chlorine, adding 0.25 kilograms of caustic soda, adding 0.25 kilograms of soda ash, adding 1 ml of chloroform, adding 5 ml of tide scent and stirring the whole mixture daily for 3 days.

### DETAILED DESCRIPTION

This is a product invented with the intention of reducing or clearing the odor in the sanitary areas e.g. toilet, sewerage, kitchen etc. It cartelizes odor to the extent of having no smell in this areas. When used on routine basis, it shall be noticed that its use keeps away flies and kill germs. It shall also be apparent that when used with other detergents, it leaves the surface spackling clean.

It’s a liquid agent which must be mixed with water for any process to take place the usual normal cleaning procedure may be applied. Keep away from food and children.

### Process of Preparation

### Ingredients.

1. ½ kg chlorine 65%
2. ¼ kg caustic soda
3. ¼ kg soda ash
4. 2 ml Chloroform
5. 5 ml tide scent

### Direction

1. Pour 10 liters of water in a container
2. Add 0.5 kgs. of chlorine in powder form & stir
3. Add 0.25 kgs. of caustic soda & stir
4. Add 0.25 kgs. of soda ash & stir
5. Add 1 ml. of chloroform & stir
6. Add 5 mls. of tide scent & stir

The composition is then stirred daily for 3 days.

1. A hygienic odor reducing agent in sanitary areas comprising:
chlorine;
caustic soda;
soda ash;
Chloroform; and
tide scent.

2. The hygienic odor reducing agent of claim 1, characterized in that, it comprises 0.5 kilograms of chlorine in powder form and constituting 65% of the solution.

3. The hygienic odor reducing agent of claim 1, characterized in that it comprises 0.25 kilograms of caustic soda in powder form, 0.25 kilograms of soda ash, 1 ml of chloroform and 5 ml of tide scent.

4. A process of preparing the hygienic odor reducing agent in sanitary areas according to claim 1, the process comprising the steps of:
pouring 10 liters of water in a container;
adding 0.5 kilograms of chlorine;
adding 0.25 kilograms of caustic soda;
adding 0.25 kilograms of soda ash;
adding 1 ml of chloroform; and
adding 5 ml of tide scent and stirring the whole mixture daily for 3 days.

5. The hygienic odor reducing agent of claim 1, wherein the agent is used as an insect repellant, odor removing and cleansing agent.
